(19) United States
(12) Patent Application Publication
(10) Pub. No.: US 2018/0085165 A1
(43) Pub. Date: Mar. 29, 2018

(54) METHOD AND COMPUTER PROGRAM PRODUCT FOR PREPLANNING ORTHOPEDIC SURGERY

(71) Applicants: Rahul Vaidya, Ann Arbor, MI (US); Andrew Jason Knapp, Essex (CA); David Wayne Schumaker, Clarkston, MI (US)

(72) Inventors: Rahul Vaidya, Ann Arbor, MI (US); Andrew Jason Knapp, Essex (CA); David Wayne Schumaker, Clarkston, MI (US)

(21) Appl. No.: 14/841,703

(22) Filed: Sep. 1, 2015

Related U.S. Application Data
(60) Provisional application No. 62/044,295, filed on Aug. 31, 2014.

(51) Int. Cl. A61B 34/10; A61B 34/00; G16H 30/40

(52) U.S. Cl. CPC A61B 34/10; A61B 34/25; A61B 6/5241; A61B 2034/104; A61B 2034/105; G16H 30/40

(57) ABSTRACT

A method of preplanning orthopedic surgery and computer program therefore. The method includes the steps of: importing an x-ray image into an electronic canvas; scaling the x-ray image to determine the pixel to distance ratio; creating on the canvas one or more moveable, rotatable trace overlays of bones or bone fragments visible in the x-ray; inserting onto the canvas one or more moveable, rotatable images of orthopedic implants; and rotating and moving the trace overlays and the images of orthopedic implants into positions depicting the planned surgical outcome.

### CROSS REFERENCE TO RELATED APPLICATIONS

This application is a non-provisional application that claims the priority of U.S. Provisional Application Ser. No. 62/044,295 filed Aug. 31, 2014.

### FIELD OF THE INVENTION

The present invention relates to orthopedic surgery and specifically to a method and computer program product for preplanning orthopedic surgery.

### BACKGROUND OF THE INVENTION

Preoperative planning of orthopedic surgery is an important part of the surgical procedure. During this process, the surgeon searches for optimal fit bone fragments and for the best technique to reconstruct the fractured bones. Preoperative planning forces the surgeon to think three-dimensionally and is thought to improve surgical precision, shorten the length of the procedure and reduce the incidence of complications. Preoperative planning also provides the surgeon with the ability to ascertain the correct orthopedic implant components and sizes to be used in the surgery, and can also be of assistance in logistic and stock management of the operation theaters.

The potential difficulty to accurately determine the magnification factor of the radiograph is one problem in analogue preoperative planning of orthopedic surgery. In addition, the use of templates of orthopedic implants with standard magnifications does not always allow for accurate correction of the magnification factor. Digital radiographs are replacing conventional radiographs to a growing extent. This allows the orthopedic surgeon to perform the planning on screen using specialized software. These applications enable the surgeon to correct the magnification factor with more accuracy and reliability. Although this might sound appealing, it is unclear what the actual advantages of digital preplanning are. The main problem is that the presently available methods and computer program products for preplanning orthopedic surgery are extremely complicated to use and are designed for very specific computer systems. What is needed is a simple to use application that allows the user to preplan orthopedic surgery on any computer device.

### SUMMARY OF THE INVENTION

The present invention is a method of preplanning orthopedic surgery. The method may include the steps of: importing an x-ray image into an electronic canvas; scaling the x-ray image to determine the pixel to distance ratio; creating on the canvas one or more moveable, rotatable trace overlays of bones or bone fragments visible in the x-ray; inserting onto the canvas one or more moveable, rotatable images of orthopedic implants; and rotating and moving the trace overlays and the images of orthopedic implants into positions depicting the planned surgical outcome.

The step of importing the x-ray image into the electronic canvas may comprise importing the x-ray from a saved computer file or importing the x-ray from a camera attached to a computer. The step of creating the trace overlays may comprise electronically tracing the perimeter of the bones or bone fragments. The step of tracing the perimeter of the bones or bone fragments may comprise a free trace of the perimeter of the bones or bone fragments or a point to point polygonal trace of the perimeter of the bones or bone fragments.

The step inserting images of orthopedic implants may comprise the steps of: selecting an orthopedic implant from a list of implants; importing an image of the selected orthopedic implant from a database; and scaling the imported image of the selected orthopedic implant using the pixel to distance scale of the x-ray image. The step of scaling the x-ray image to determine the pixel to distance ratio may comprise manual scaling of the image or automatic scaling using an image of a scale embedded into the x-ray. The image of a scale embedded into the x-ray may be created by an x-ray opaque object of known dimensions placed the field of the x-ray as it is created.

### BRIEF DESCRIPTION OF THE DRAWINGS

FIG. 1A illustrates a computing device in the form of a computer 110, which is capable of performing one or more computer-implemented steps in practicing the method aspects of the present invention;

FIG. 1B depicts the process flow diagram for one embodiment of the present computer program product;

FIG. 1C is a flow chart of one embodiment of the initiation of the program and importation of the orthopedic image is depicted in.

FIG. 1D is a flow chart of one embodiment of the add x-ray procedure of FIG. 1C;

FIG. 1E is a flow chart of one embodiment of a first portion of an autoscaling code/procedure;

FIG. 1F is a flow chart of one embodiment of a second portion of an autoscaling code/procedure;

FIG. 1G is a flow chart of one embodiment of a third portion of an autoscaling code/procedure;

FIG. 1H is a flow chart of one embodiment of the free trace procedure;

FIG. 1I is a flow chart of one embodiment of the polygon trace procedure;

FIG. 1J is a flow chart of one embodiment of the move object procedure;

FIG. 1K is a flow chart of one embodiment of the rotate object procedure;

FIG. 1L is a flow chart of one embodiment of the save project procedure;

FIG. 1M is a flow chart of one embodiment of the add tool procedure;

FIG. 1N is a flow chart of one embodiment of the load project procedure;

FIG. 1O is a flow chart of one embodiment of the get distance procedure;

FIG. 1P is a flow chart of one embodiment of the get distance procedure;

FIG. 1Q is a flow chart of one embodiment of the get distance procedure;

FIG. 1R is a flow chart of one embodiment of the to calculate the length of a line procedure;

FIG. 2 is a screen shot of the GUI opening screen where the user can choose to load a digital x-ray image from a file or a camera, or to load a saved project;

FIG. 3 is a screen shot of the GUI when user has chosen to load an x-ray from a file;

FIG. 4 is a screen shot of the GUI when user has chosen to load an x-ray from the gallery folder;

FIG. 5 is a screen shot of the GUI when user has chosen to load an x-ray from the camera subfolder;

FIG. 6 is a screen shot of the GUI when user has chosen to load a specific x-ray from a specific image file;

FIG. 7 is a screen shot of the GUI when user has chosen to use the auto scaling option;

FIG. 8 is a screen shot of the GUI when user has chosen to zoom the image to better view the bones for tracing;

FIG. 9 is a screen shot of the GUI when user has chosen to create a polygon trace of a bone segment;

FIG. 10 is a screen shot of the GUI when user has created two more polygon traces of bone segments;

FIG. 11 is a screen shot of the GUI when user selected the XRAY 1 layer and has used the show/hide button to hide the x-ray;

FIG. 12 is a screen shot of the GUI when user has selected the new tool button to import an implant/tool image;

FIG. 13 is a screen shot of the GUI when user has moved the bone plate into place adjacent the bone overlays;

FIG. 14 is a screen shot of the GUI when user has moved the 26 mm cortical screw into place to fix the plate to one of the bone overlays;

FIG. 15 is a screen shot of the GUI when user has imported and placed the 18 mm cortical screw;

FIG. 16 is a screen shot of the GUI when user has again used the distance feature to get the length of screw needed for the top of the plate;

FIG. 17 is a screen shot of the GUI when user has imported and placed the final, 16 mm cortical screw;

FIG. 18 is a screen shot of the GUI showing the completed virtual surgery further zoomed to 191%;

FIG. 19 is a screen shot of the GUI when the user has used the show/hide button to bring the original x-ray back into view under the bone trace and orthopedic tool image overlays;

FIG. 20 is a screen shot of the GUI when the user has chosen the save button to save the project;

### DETAILED DESCRIPTION OF THE PREFERRED EMBODIMENTS

The present invention will now be described more fully hereinafter with reference to the accompanying drawings, in which preferred embodiments of the invention are shown. This invention may, however, be embodied in many different forms and should not be construed as limited to the embodiments set forth herein. Rather, these embodiments are provided so that this disclosure will be thorough and complete, and will fully convey the scope of the invention to those skilled in the art. Like numbers refer to like elements throughout.

Embodiments of the present invention are described herein in the context of a system of computers, servers, and software. Those of ordinary skill in the art will realize that the following embodiments of the present invention are only illustrative and are not intended to be limiting in any way. Other embodiments of the present invention will readily suggest themselves to such skilled persons having the benefit of this disclosure.

In the interest of clarity, not all of the routine features of the implementations described herein are shown and described. It will, of course, be appreciated that in the development of any such actual implementation, numerous implementation-specific decisions must be made in order to achieve the developer's specific goals, such as compliance with application- and business-related constraints, and that these specific goals will vary from one implementation to another and from one developer to another. Moreover, it will be appreciated that such a development effort might be complex and time-consuming, but would nevertheless be a routine undertaking of engineering for those of ordinary skill in the art having the benefit of this disclosure.

In accordance with embodiments of the present invention, the components, process steps, and/or data structures may be implemented using various types of operating systems, computing platforms, computer programs, and/or general purpose machines. In addition, after having the benefit of this disclosure, those of ordinary skill in the art will recognize that devices of a less general purpose nature, such as hardwired devices, field programmable gate arrays (FPGAs), application specific integrated circuits (ASICs), or the like, may also be used without departing from the scope and spirit of the inventive concepts disclosed herein.

The computer program product for preplanning orthopedic surgery according to the present invention, may be a computerized system that requires the performance of one or more steps to be carried out on or in association with a computerized device. A person of skill in the art will appreciate that the computerized device may include, but not be limited to, a server, a computer (i.e., desktop computer, laptop computer, netbook, tablet, or any machine having a processor), a dumb terminal that provides an interface with a computer or server, a personal digital assistant, mobile communications device, such as a mobile phone, smart phone, or other similar device that provides computer or quasi-computer functionality, a mobile reader, such as an electronic document viewer, which provides reader functionality that may be enabled, through either internal components or connecting to an external computer, server, or global communications network (such as the Internet), to take direction from or engage in processes which are then delivered to the mobile reader. It should be readily apparent to those of skill in the art, after reviewing the materials disclosed herein, that other types of devices, individually or in conjunction with an overarching architecture, associated with an internal or external system, may be utilized to provide the “computerized” environment necessary for the process step to be carried out in a machine/system/digital environment. It should be noted that the method aspects of the present invention are preferably computer-implemented methods and, more particularly, at least one step is preferably carried out using a computerized device.

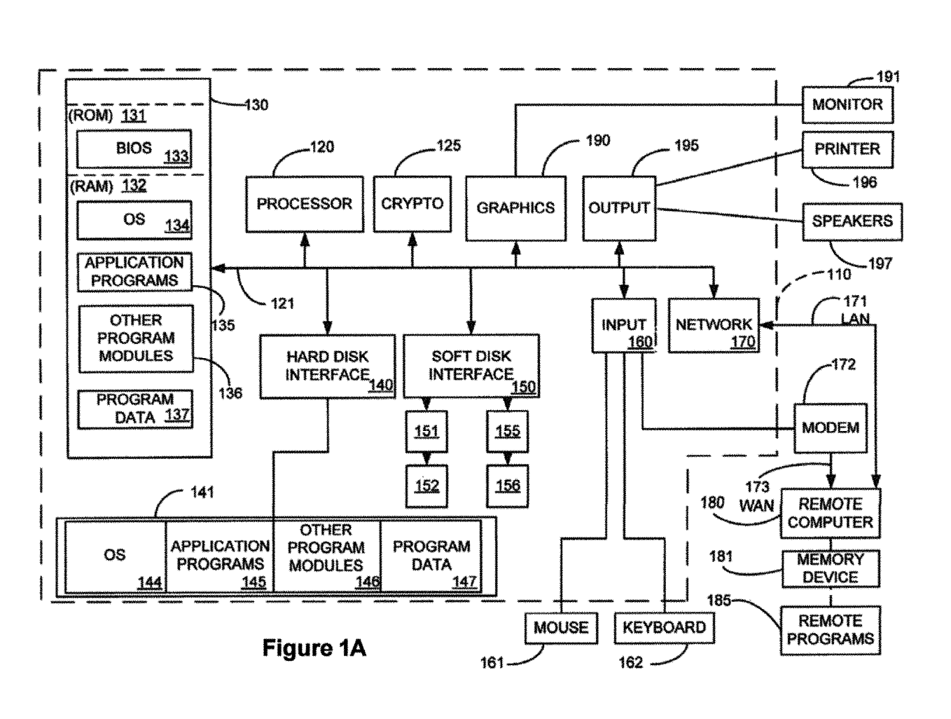

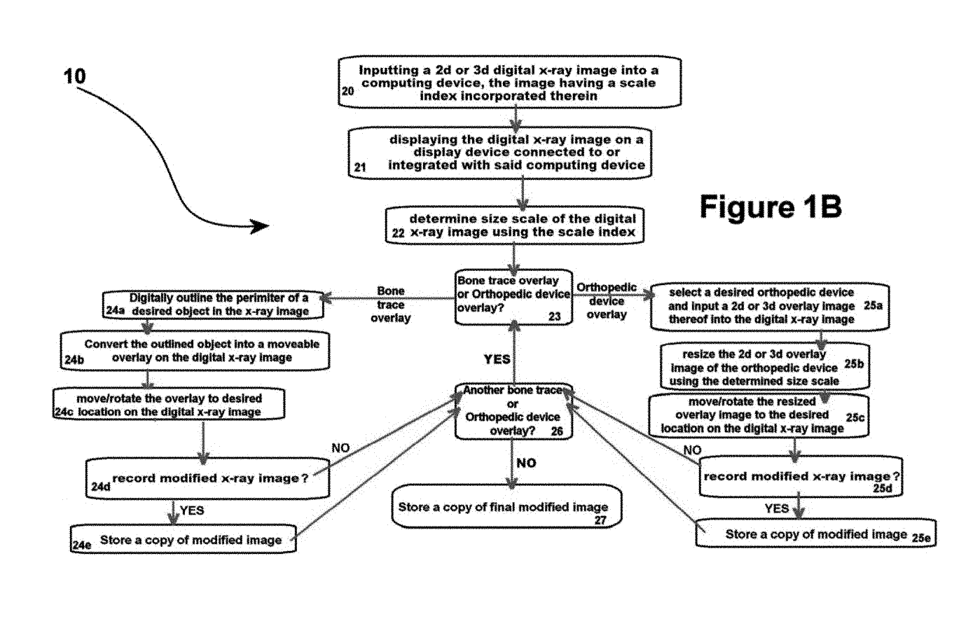

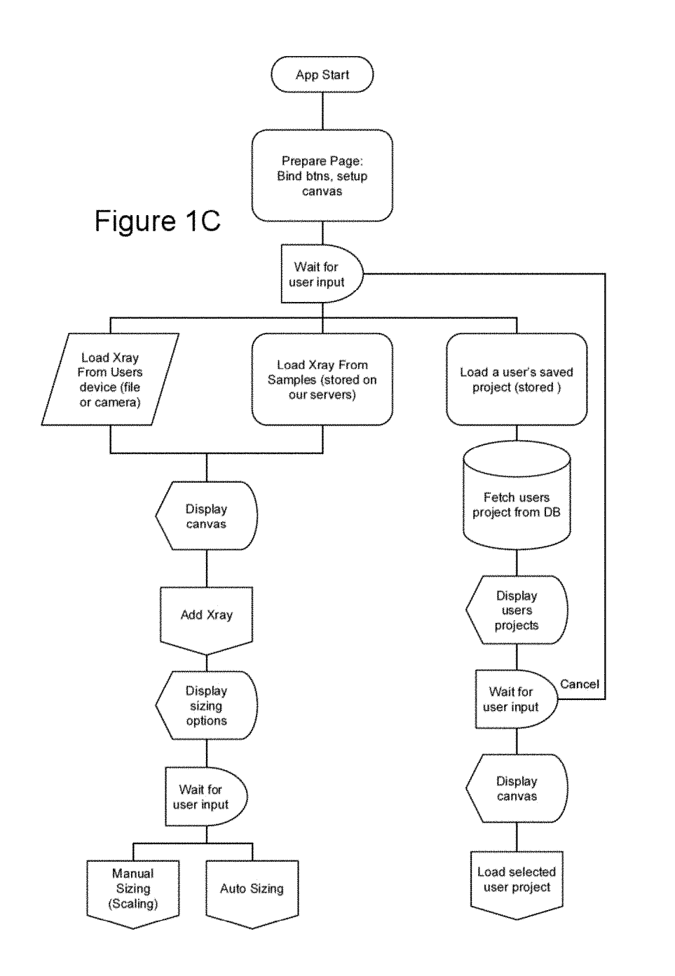

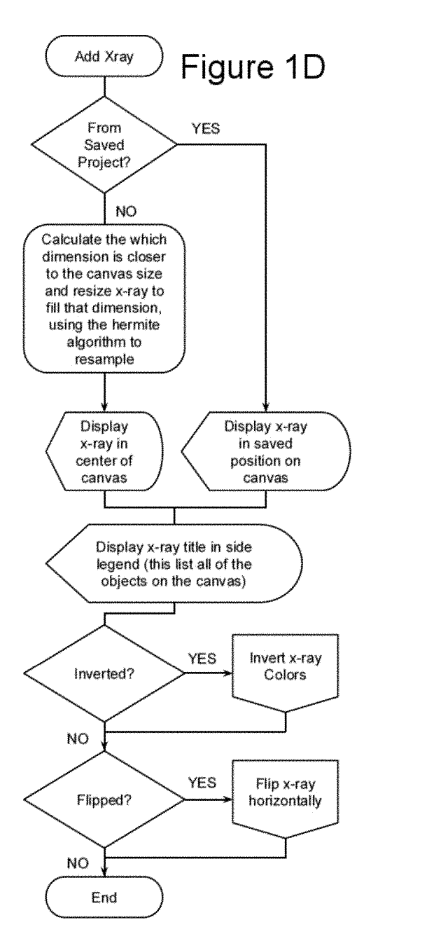

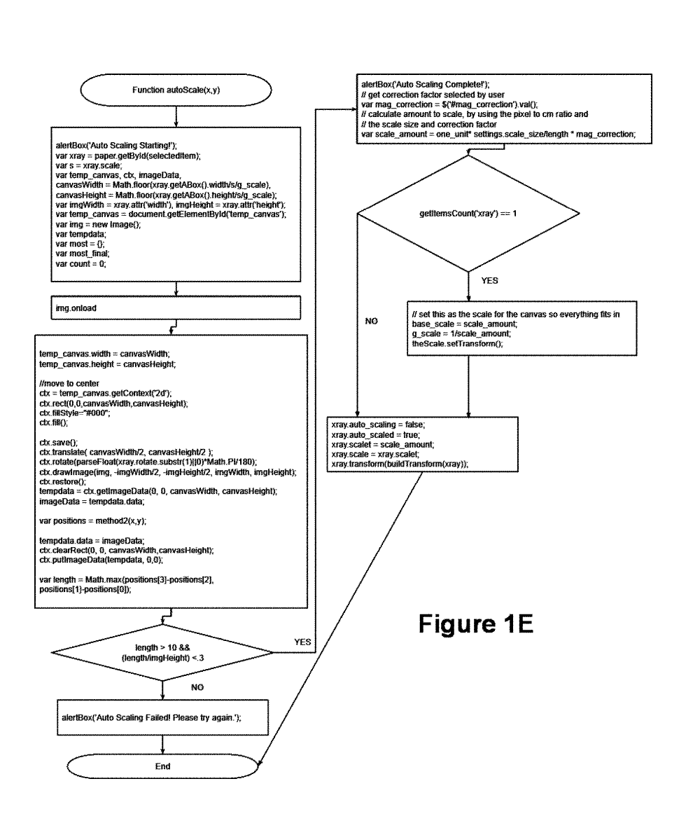

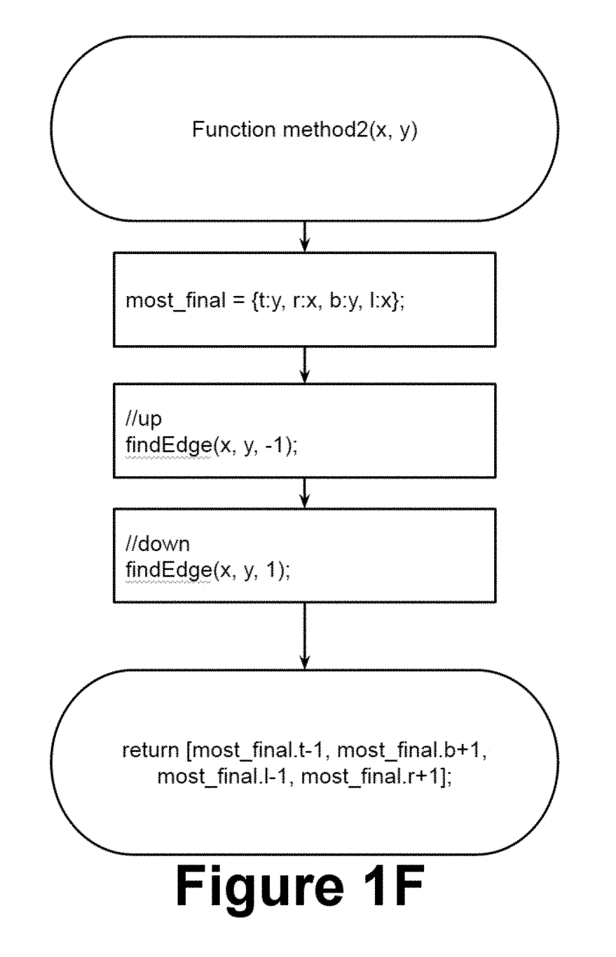

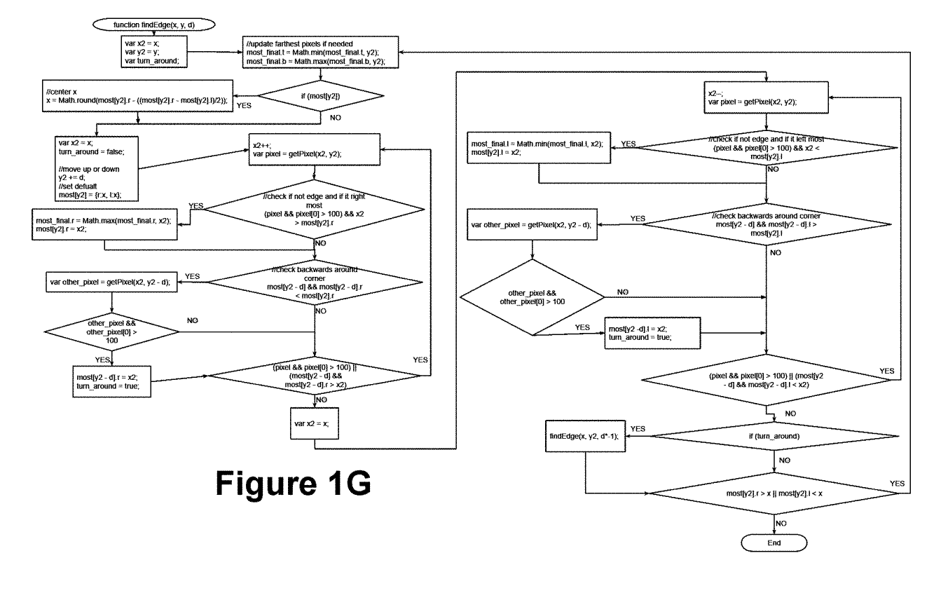

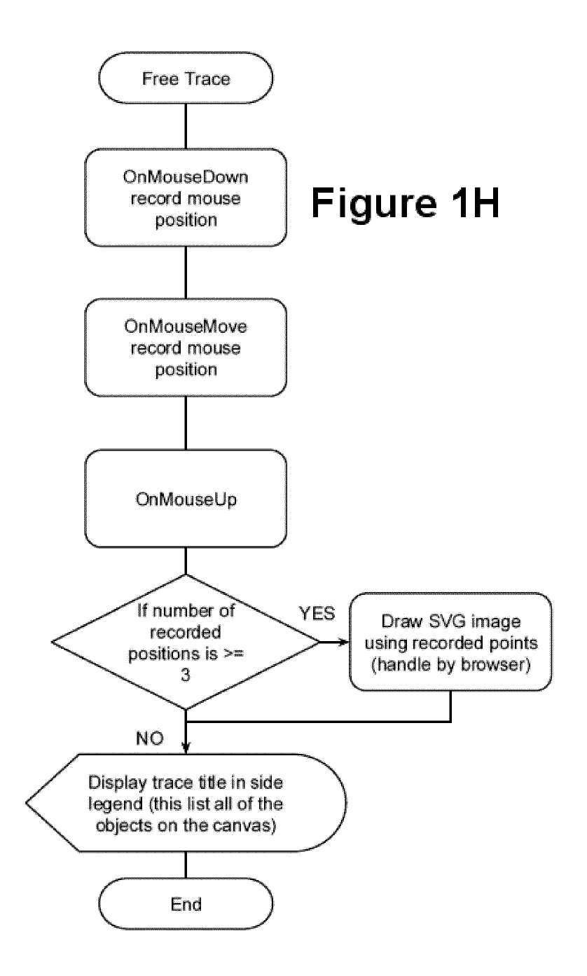

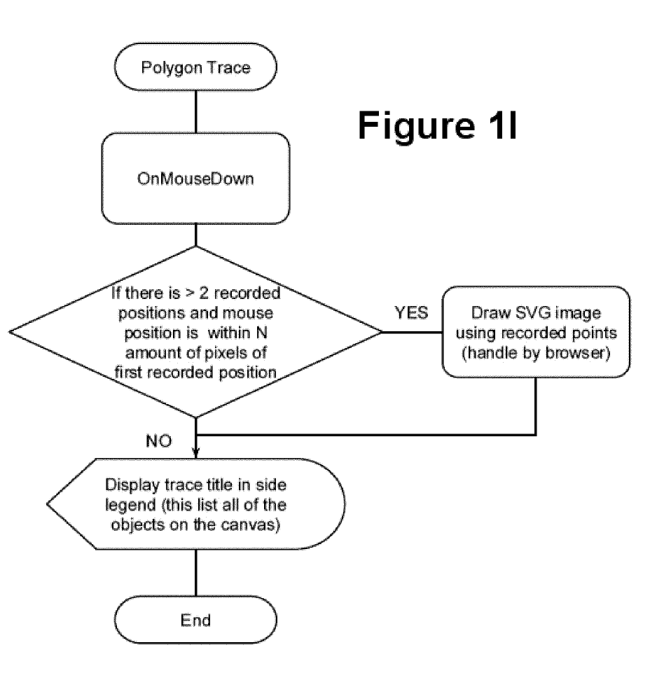

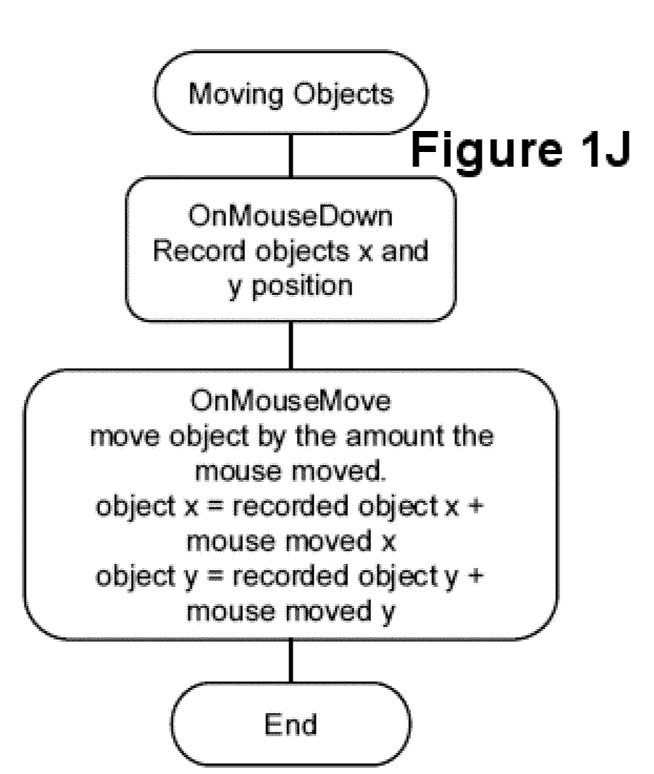

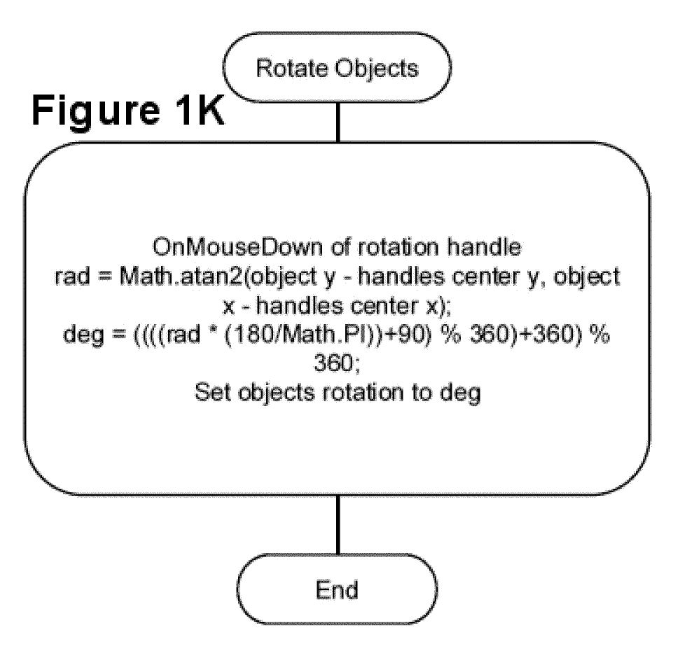

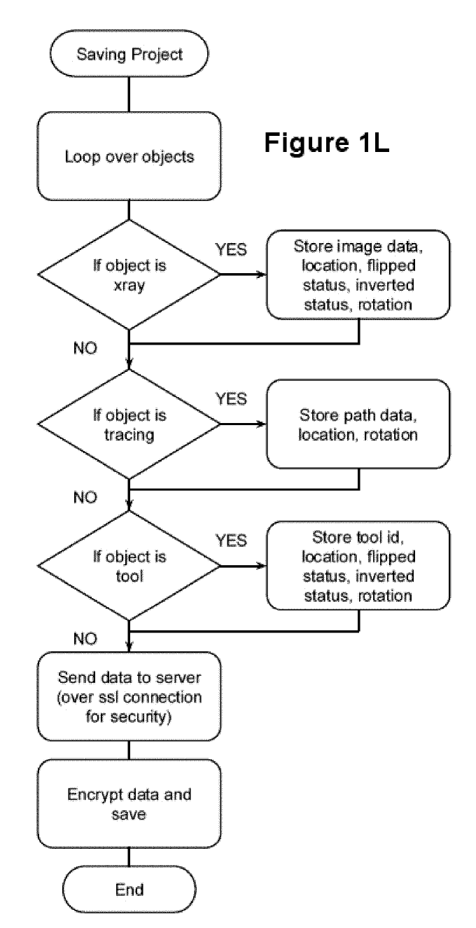

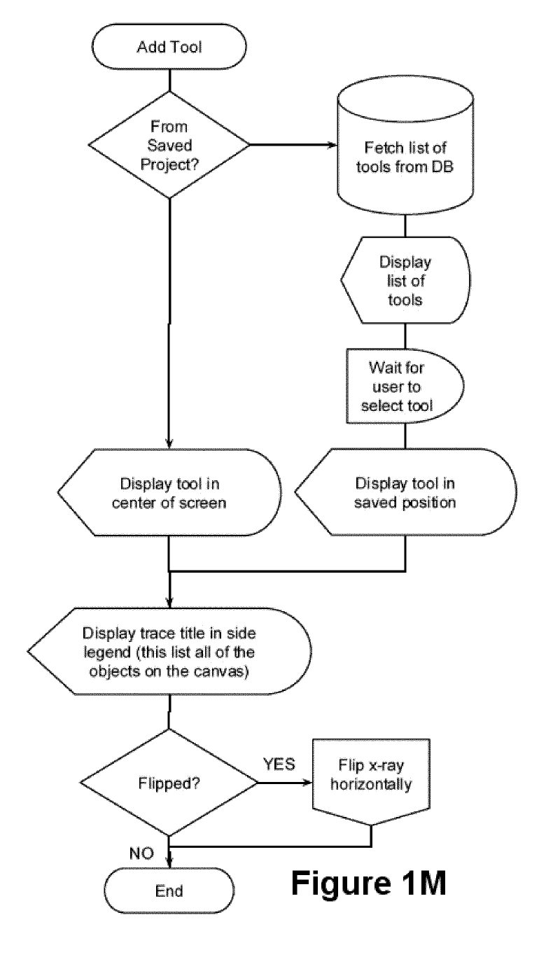

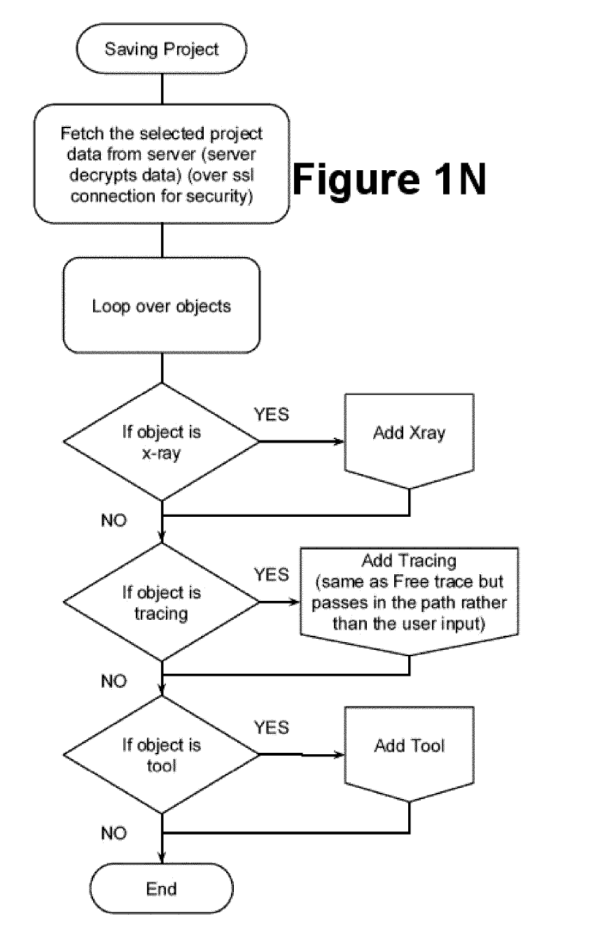

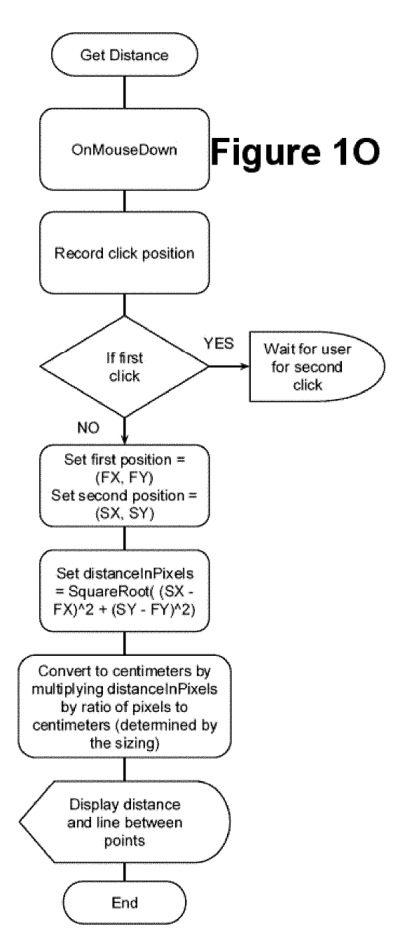

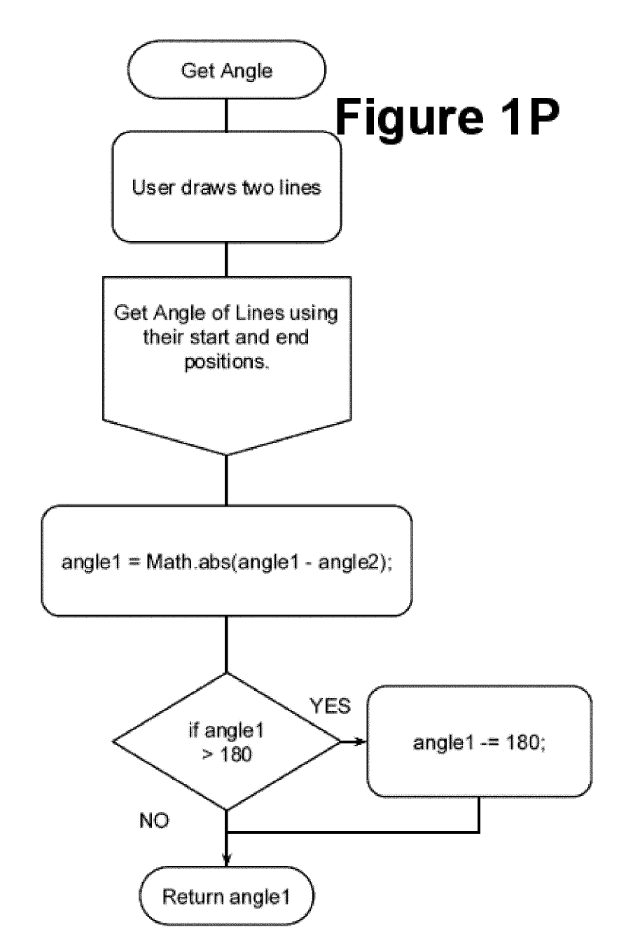

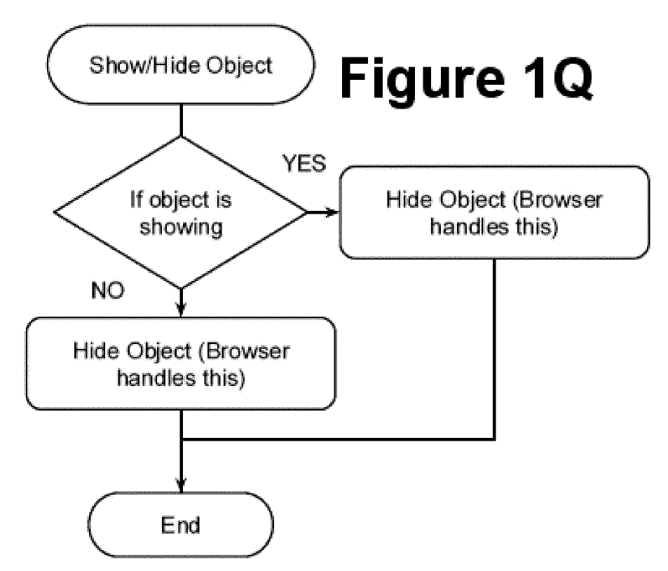

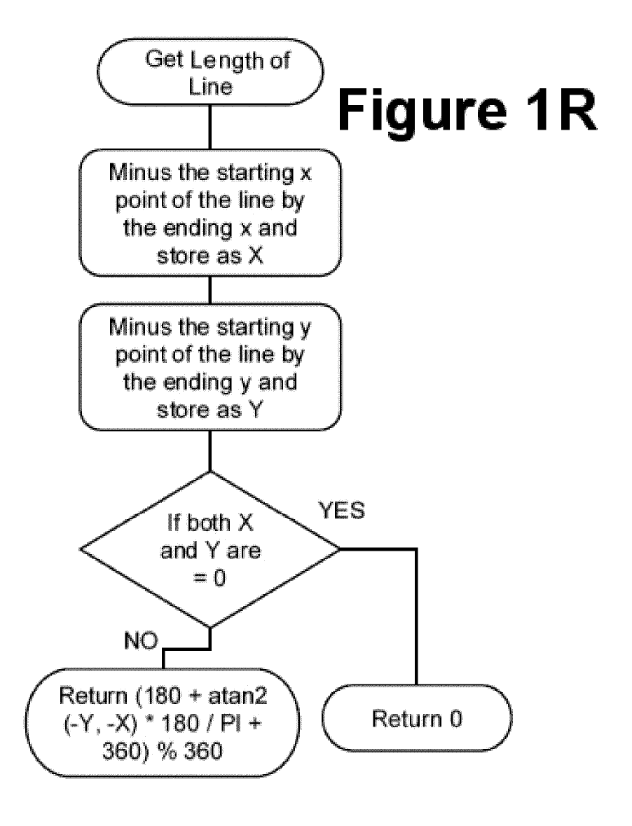

FIG. 1A illustrates a computing device in the form of a computer 110, which is capable of performing one or more computer-implemented steps in practicing the method aspects of the present invention. Components of the computer 110 may include, but are not limited to a processing unit 120, a system memory 130, and a system bus 121 that couples various system components including the system memory to the processing unit 120. The system bus 121 may be any of several types of bus structures including a memory bus or memory controller, a peripheral bus, and a local bus using any of a variety of bus architectures. By way of example, and not limitation, such architectures include Industry Standard Architecture (ISA) bus, Micro Channel Architecture (MCA) bus, Enhanced ISA (EISA) bus, Video Electronics Standards Association (VESA) local bus, and Peripheral Component Interconnect (PCI).

The computer 110 may also include a cryptographic unit 125. Briefly, the cryptographic unit 125 has a calculation function that may be used to verify digital signatures, calculate hashes, digitally sign hash values, and encrypt or decrypt data. The cryptographic unit 125 may also have a protected memory for storing keys and other secret data. In other embodiments, the functions of the cryptographic unit may be instantiated in software and run via the operating system.

A computer 110 typically includes a variety of computer readable media. Computer readable media can be any available media that can be accessed by a computer 110 and includes both volatile and nonvolatile media, removable and non-removable media. By way of example, and not limitation, computer readable media may include computer storage media and communication media. Computer storage media includes volatile and nonvolatile, removable and non-removable media implemented in any method or technology for storage of information such as computer readable instructions, data structures, program modules or other data. Computer storage media includes, but is not limited to, RAM, ROM, EEPROM, FLASH memory or other memory technology, CD-ROM, digital versatile disks (DVD) or other optical disk storage, magnetic cassettes, magnetic tape, magnetic disk storage or other magnetic storage devices, or any other medium which can be used to store the desired information and which can be accessed by a computer 110. Communication media typically embodies computer readable instructions, data structures, program modules or other data in a modulated data signal such as a carrier wave or other transport mechanism and includes any information delivery media. The term “modulated data signal” means a signal that has one or more of its characteristics set or changed in such a manner as to encode information in the signal. By way of example, and not limitation, communication media includes wired media such as a wired network or direct-wired connection, and wireless media such as acoustic, radio frequency, infrared and other wireless media. Combinations of any of the above should also be included within the scope of computer readable media.

The system memory 130 includes computer storage media in the form of volatile and/or nonvolatile memory such as read only memory (ROM) 131 and random access memory (RAM) 132. A basic input/output system 133 (BIOS), containing the basic routines that help to transfer information between elements within computer 110, such as during start-up, is typically stored in ROM 131. RAM 132 typically contains data and/or program modules that are immediately accessible to and/or presently being operated on by processing unit 120. By way of example, and not limitation, FIG. 1A illustrates an operating system (OS) 134, application programs 135, other program modules 136, and program data 137.

The computer 110 may also include other removable/non-removable, volatile/nonvolatile computer storage media. By way of example only, FIG. 1A illustrates a hard disk drive 141 that reads from or writes to non-removable, nonvolatile magnetic media, a magnetic disk drive 151 that reads from or writes to a removable, nonvolatile magnetic disk 152, and an optical disk drive 155 that reads from or writes to a removable, nonvolatile optical disk 156 such as a CD ROM or other optical media. Other removable/non-removable, volatile/nonvolatile computer storage media that can be used in the exemplary operating environment include, but are not limited to, magnetic tape cassettes, flash memory cards, digital versatile disks, digital video tape, solid state RAM, solid state ROM, and the like. The hard disk drive 141 is typically connected to the system bus 121 through a non-removable memory interface such as interface 140, and magnetic disk drive 151 and optical disk drive 155 are typically connected to the system bus 121 by a removable memory interface, such as interface 150.

The drives, and their associated computer storage media discussed above and illustrated in FIG. 1A, provide storage of computer readable instructions, data structures, program modules and other data for the computer 110. In FIG. 1A, for example, hard disk drive 141 is illustrated as storing an OS 144, application programs 145, other program modules 146, and program data 147. Note that these components can either be the same as or different from OS 134, application programs 135, other program modules 136, and program data 137. The OS 144, application programs 145, other program modules 146, and program data 147 are given different numbers here to illustrate that, at a minimum, they may be different copies. A user may enter commands and information into the computer 110 through input devices such as a keyboard 162 and cursor control device 161, commonly referred to as a mouse, trackball or touch pad. Other input devices (not shown) may include a microphone, joystick, game pad, satellite dish, scanner, touch screen, or the like. These and other input devices are often connected to the processing unit 120 through a user input interface 160 that is coupled to the system bus, but may be connected by other interface and bus structures, such as a parallel port, game port or a universal serial bus (USB). A monitor 191 or other type of display device is also connected to the system bus 121 via an interface, such as a graphics controller 190. In addition to the monitor, computers may also include other peripheral output devices such as speakers 197 and printer 196, which may be connected through an output peripheral interface 195.

The computer 110 may operate in a networked environment using logical connections to one or more remote computers, such as a remote computer 180. The remote computer 180 may be a personal computer, a server, a router, a network PC, a peer device or other common network node, and typically includes many or all of the elements described above relative to the computer 110, although only a memory storage device 181 has been illustrated in FIG. 1A. The logical connections depicted in FIG. 1A include a local area network (LAN) 171 and a wide area network (WAN) 173, but may also include other networks. Such networking environments are commonplace in offices, enterprise-wide computer networks, intranets and the Internet.

When used in a LAN networking environment, the computer 110 is connected to the LAN 171 through a network interface or adapter 170. When used in a WAN networking environment, the computer 110 typically includes a modem 172 or other means for establishing communications over the WAN 173, such as the Internet. The modem 172, which may be internal or external, may be connected to the system bus 121 via the user input interface 160, or other appropriate mechanism. In a networked environment, program modules depicted relative to the computer 110, or portions thereof, may be stored in the remote memory storage device. By way of example, and not limitation, FIG. 1A illustrates remote application programs 185 as residing on memory device 181.

The communications connections 170 and 172 allow the device to communicate with other devices. The communications connections 170 and 172 are an example of communication media. The communication media typically embodies computer readable instructions, data structures, program modules or other data in a modulated data signal such as a carrier wave or other transport mechanism and includes any information delivery media. A “modulated data signal” may be a signal that has one or more of its characteristics set or changed in such a manner as to encode information in the signal. By way of example, and not limitation, communication media includes wired media such as a wired network or direct-wired connection, and wireless media such as acoustic, RF, infrared and other wireless media. Computer readable media may include both storage media and communication media.

According to an embodiment of the present invention, a computer program product 10 is disclosed, which may be capable of preplanning an orthopedic surgical technique. A computer program product 10, according to an embodiment of the present invention, will be described in further detail as a project that is creatable using, for example, but not intended as a limitation, one or more of Microsoft's Visual C# .NET development environment, Java Script, HTML (HyperText Markup Language), and/or CSS (Cascading Styles Sheets). Such a computer program product 10 would be suitable for execution on a computer 110 having, for example, but not intended as a limitation, one of Microsoft's Windows family, Apple's Mac OSX family, or Google's Android mobile family operating system of operating systems loaded into memory 134. A person having skill in the art, after having the benefit of this disclosure would recognize that many other development platforms might be used to create a computer program product 10, which may be executable with many other operating systems, but that still embody the present invention. As such, the following disclosure is provided merely for explanatory purposes and should in no way limit the present invention to computer program products 10 that are created using the aforementioned development platform or for use with the aforementioned operating systems.

Viewed most generally, the program 10 of the present invention is a system that allows an orthopedic surgeon to preplan an orthopedic surgery. FIG. 1B depicts the process flow diagram for one embodiment of the present computer program product 10. At step 20, the program imports 2d or 3d orthopedic images, such as x-rays or X-ray computed tomography. The images may have thereon a scale measuring indicator. A flow chart of one embodiment of the initiation of the program and importation of the orthopedic image is depicted in FIG. 1C. A flow chart of one embodiment of the add x-ray procedure of FIG. 1C is depicted in FIG. 1D.

The scaling indicator is preferably a round disk made of an x-ray opaque material, such as for instance metal (aluminum, steel, titanium, etc). The disk is of a known dimension, such as for instance 3 cm or 4 cm, etc. The disk may have a pattern carved there through forming an x-ray translucent area. The pattern may be for instance an L or an R to indicate which appendage is being x-rayed. The disk should be supported on a stand of some sort to raise it to the level of the bone(s) being x-rayed. This insures more accurate scaling of the objects in the x-ray. The stand should preferably be x-ray translucent.

The image may be imported by being captured by a camera attached to or integrated with the computer 110. For example, the image may be imported by taking of a picture of a displayed x-ray on another screen, or displayed as a printed film on a x-ray viewing box. The image may also be directly imported by downloading the image from a storage medium or directly from a digital x-ray imaging machine. In general, the image data is loaded from a camera or file system. The program 10 may resize the image to fit the screen and then display the image. Calculate the dimensions of the image vs the screen to fit the image in.

A=(image width*screen height)/image height).

B=(image height*screen width)/image width).

If A is greater than screen width then scale image to screen width otherwise scale image to screen height. Next the program store info related to the image with it, for example position, scaling, index, id, rotation, etc . . . If the image is coming from a saved project, the image related info is restored into memory. Then, the image is added to or displayed on the screen, step 21 of FIG. 1B. A flow chart of one embodiment of the add x-ray procedure is depicted in FIG. 1D.

At step 22, the program 10 allows the user to choose manual scaling of the image by the user or autoscaling using the image of the scale measuring indicator. For auto scaling, the user clicks a point (x,y) within the scaling index object and the x-y coordinates are passed to The Function with x,y, and up as the direction.

Note: direction is represented as 1 (up) or −1 (down), so that it can just be added to the y coordinate to move it the desired direction and direction can be reversed by multiplying it by −1.

### The Function (Params x, y, Direction)

We setup a variable that will keep track of our furthest points in each direction (up, down, left, right) and another variable that keeps track of each horizontal line's left and right most point. Then it enters a loop which increments either upwards or downwards from a give point.

### In the Loop (LOOP A):

First we update the highest or lowest point if its greater than our last saved point. Next if the lines left and right most point is set we set the x position to be centered between them (so right-((right-left)/2) and round) Then we enter another loop which increments right.

In that Loop (LOOP B):

First we get the pixel and check if it is white (or gray) and if it is further right than the current furthest for that line, if it is then we update the furthest for that line, and then check if it is also our furthest over all in that direction and update if it is.

Second if the previous line right most pixel is already set and it is less than the current lines, then we get the pixel in the previous line that lines up with the current pixel and if it is white (or gray) then we update the previous lines right most pixel and set a variable (lets call it) VAR1 to true;

Finally in order to check to continue or exit the loop rightward we check if the pixel is white (or gray) or if the previous lines right most pixel is greater than our current x position, and if it is we continue the loop otherwise we exit it
LOOP B escaped
LOOP C
Next we re-run the previous loop (LOOP B) but leftward;
LOOP C escaped

Now we check if VAR1 is true and if so we call The Function with our current x position and the current y position, and the reversed directions (direction*−1) . . . Thus, the function is calling itself from within itself.

Finally for LOOP A we check if the current line's left or right most point is greater than its starting x point if it is we continue looping up or downwards otherwise we exit the loop. (The reason for checking if that line's left or right most point is greater than its starting x point is to see if we have hit the top or bottom)

### End of The Function

Now we simply subtract the highest point from the lowest and the left most point from the right and uses the larger dimension for our pixel to length (i.e. cm, mm, inches, etc.) scaling. A flow chart of one embodiment of the autoscaling code/procedure is depicted in FIGS. 1E, 1F and IG.

Once the scaling index is determined, the user may now choose (step 23) to create a trace overlay (possibly of a bone or bone fragment) or import an overlay image of an orthopedic implant such as a screw, plate, rod, etc.

If the user chooses a trace overlay, the program allows for digital outlining of the perimeter of the object being traced, see step 24a. The program 10 allows for two different methods to trace the object. The first method is a free trace. The user touches a start position and the coordinates are add to a list of positions of the path. As the user continuously traces the object, new coordinates are also added to the list. When the user stops tracing (lifts the stylus or finger from a touch screen, or releases the mouse button), the path is then closed. The tracing is filled with a color and the tracing is converted to a moveable overlay on the digital x-ray image (step 24b). The moveable trace overlay is added to the list of objects in the display image. A flow chart of one embodiment of the free trace procedure is depicted in FIG. 1H.

The second method is a polygon trace. The user clicks a starting position on the perimiter of the object, the coordinates of the starting position are added to the list of positions within the path of the trace. Next the user moves the cursor to another position along the perimeter and again clicks (or touches the touch screen) and the coordinates of the new position are added to the list of positions within the path of the trace. When the position of the most recent point is within a defined distance from the starting position, the path is closed. The tracing is filled with a color and the tracing is converted to a moveable overlay on the digital x-ray image (step 24b). The moveable trace overlay is added to the list of objects in the display image. A flow chart of one embodiment of the polygon trace procedure is depicted in FIG. 1I.

Once the overlay is created, it may be moved and/or rotated to a desired position on the digital x-ray image, see step 24c. A flow chart of one embodiment of the move object procedure is depicted in FIG. 1J. A flow chart of one embodiment of the rotate object procedure is depicted in FIG. 1K.

At this point (or alternatively anywhere along the way), a copy of the digital x-ray and any/all overlay images may be saved to be used in, for instance a slide show of the planing steps in the full surgical procedure, see steps 24d and 24e. The entire project including all slides and their overlays can be save to permanent storage as a project. A flow chart of one embodiment of the save project procedure is depicted in FIG. 1L. The procedure for saving a slide is the same as that for saving a project, but the data is not saved to permanent storage.

Then, the user decides if there are additional bone traces or orthopedic implant overlays to be created (see step 26). If there is/are additional overlay image(s) to be added to the digital x-ray, the user again chooses (step 23) to create a trace overlay (possibly of a bone or bone fragment) or import an overlay image of an orthopedic implant such as a screw, plate, rod, etc.

If the user chooses to import an overlay image of an orthopedic implant (also referred to as a tool), the specific implant is chosen from a list of orthopedic implant devices, and a 2d or 3d overlay image of the orthopedic implant device is imported into the digital x-ray, see step 25a. The orthopedic implant overlay image is then resized (see step 25b) using the scaling index determined in step 22 and converted to a moveable overlay on the digital x-ray image. The moveable orthopedic implant overlay is added to the list of objects in the display image. A flow chart of one embodiment of the add tool procedure is depicted in FIG. 1M. The computer program 10 may also allow for changing the tool to larger or smaller versions of the tool (i.e. a 7 mm screw to an 8 mm or 6 mm screw) as desired or needed by a separate function.

Once the moveable orthopedic implant overlay is created, it may be moved and/or rotated to a desired position on the digital x-ray image, see step 25c. Again, as with the bone tracings, a flow chart of one embodiment of the move object procedure is depicted in FIG. 1J, and a flow chart of one embodiment of the rotate object procedure is depicted in FIG. 1K.

At this point (or alternatively anywhere along the way), a copy of the digital x-ray and any/all overlay images may be saved to be used in, for instance a slide show of the planing steps in the full surgical procedure, see steps 25d and 25e. Again, the entire project including all slides and their overlays can be save to permanent storage as a project. A flow chart of one embodiment of the save project procedure is depicted in FIG. 1L. The procedure for saving a slide is the same as that for saving a project, but the data is not saved to permanent storage.

Once again, the user decides if there are additional bone traces or orthopedic implant overlays to be created (see step 26). If there are no additional overlay images to be added to the digital x-ray, the user has the option to save the final image, step 27.

The orthopedic implant device images may be stored in local memory or hard disk form, optical disks, etc. The images may also be stored remotely such as on a server connected to the computer 110 via hard wired, wirelessly or over the internet.

Once the preplanning is completed, the images saved along the way may be used to make a slide show, used in presentations, imported into programs like Power Point, or converted to a PDF.

It should be noted that while the steps 24a-24e and 25a-25e in FIG. 1B are listed sequentially, the user may create multiple overlays and then move/rotate overlays as desired. The user may delete overlays and create new overlays at any time. The program may display a list of images and overlays that compose the overall image such that any individual component may be chosen by clicking on the desired component in the list, and once the component is selected it may be moved, rotated or deleted. Overlay objects in the image may be moved forward or backward so as to prioritize the visibility of the objects in the overall image.

Adding Slides: The user clicks the add slide button and all of the objects (x-rays, tracings, implants, . . . ) are looped through and we record each items info that will be needed to recreate that slide (position, scaling, rotation, visibility, inverted)

### Loading Slide

When the user clicks on the desired slide in the list of slides, the recorded info for all of the objects of that slide are appended back on to each item. Returning it to the state at which it was in the slide.

### Save Project

When the user saves a project, all of the objects (x-rays, tracings, implants, etc.) are looped through and each items info that will be needed to recreate the item are recorded as is done for a slide, but this time we also record the x-rays source data (the image), and the trace's paths, and the implants used. Then we also add the slides. Then record all of this data to the information storage system of the computer 110 (local or remote storage). See FIG. 1L.

### Load Project

When the user selects the desired file from the device the info is retrieved all of the objects (x-rays, bone tracings, implant images, etc.) are looped through and passed in to their corresponding function (so xray would passed into the add image function, bone traces would passed into the add trace function, and implant images would passed into the add implants function), but this time the items related info would be passed in with it instead of the users input. All the slide data is also loaded back into memory. A flow chart of one embodiment of the load project procedure is depicted in FIG. 1N.

The program 10 may also have a feature that allows the user to get the distance between two points on the image. The user clicks (or touches) the starting point of the distance they would like to measure and then the user click the end point of the distance. The distance between the two points is then calculated in pixels and using the scaling index, the number of pixels is converted to distance (e.g. pixels to cm using the pixel per cm ratio. This feature can help the user to determine, for example, the length of screw needed, or the length of rod or length of plate needed, etc. A flow chart of one embodiment of the get distance procedure is depicted in FIG. 1O.

The program 10 may also have a feature that allows the user to get the angle between two lines. A flow chart of one embodiment of the get distance procedure is depicted in FIG. 1P.

The program 10 may also have a feature that allows the user to hide or show any object in the list of objects of the image. A flow chart of one embodiment of the get distance procedure is depicted in FIG. 1Q.

The program 10 may also have a feature that allows the user to calculate the length of a line. A flow chart of one embodiment of the to calculate the length of a line procedure is depicted in FIG. 1R.

Other functions that the program 10 may also have are the ability to mirror image the x-ray and to convert the xray to a negative image.

In another embodiment, the images of the orthopedic implants may be hyperlinked in such a way as to allow the user to access the device manufacturers web pages related to the specific device, allowing the user to obtain useful information about the device that they intend to use in the surgery that is being preplanned.

In yet another embodiment, the present computer program may be linked to the user's institution's electronic inventory of orthopedic implants or alternatively be designed to incorporate an inventory feature therein. In either event, the image of the implant device may be hyperlinked to the electronic inventory and the number of available implant device of the type depicted in the image may be displayed for the user. Furthermore, the image of the implant device may be linked to a sales site or representative to provide the user with a simple method to purchase additional items depicted in the image. This inventory and/or sales function will allow institutions to carry less excess inventory of implants while ensuring that the items needed for any specific surgery will be available.

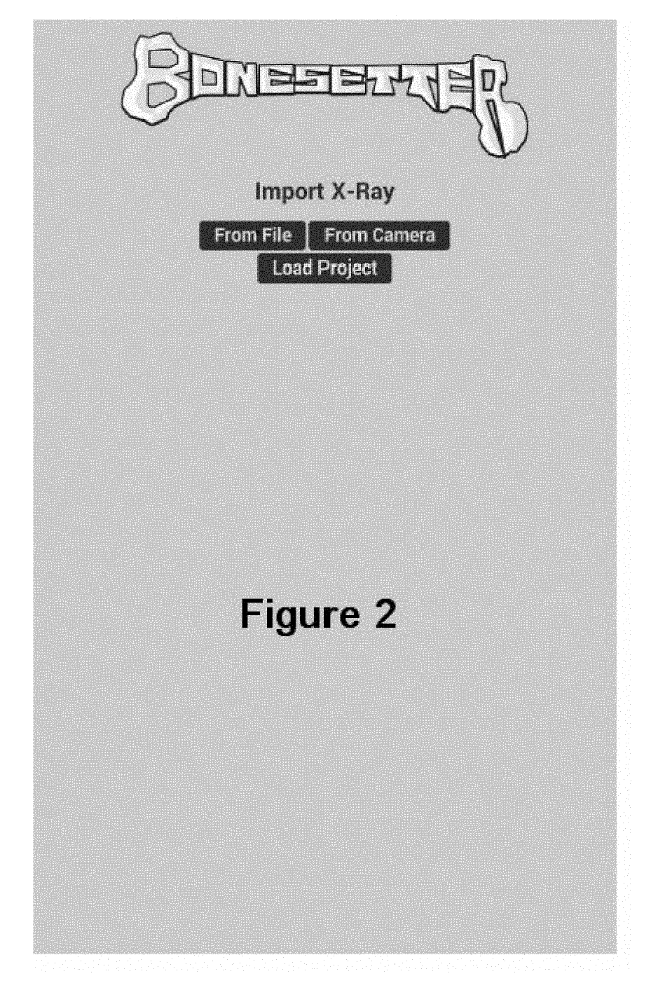

Turning now to FIGS. 2-20, there are depicted therein screen shots of the user interface and display screen of an embodiment of the computer program 10 of the present invention in action. The figures will be discussed in relation to the features and interface controls they depict.

FIG. 2 is a screen shot of the GUI opening screen where the user can choose to load a digital x-ray image from a file or a camera, or to load a saved project.

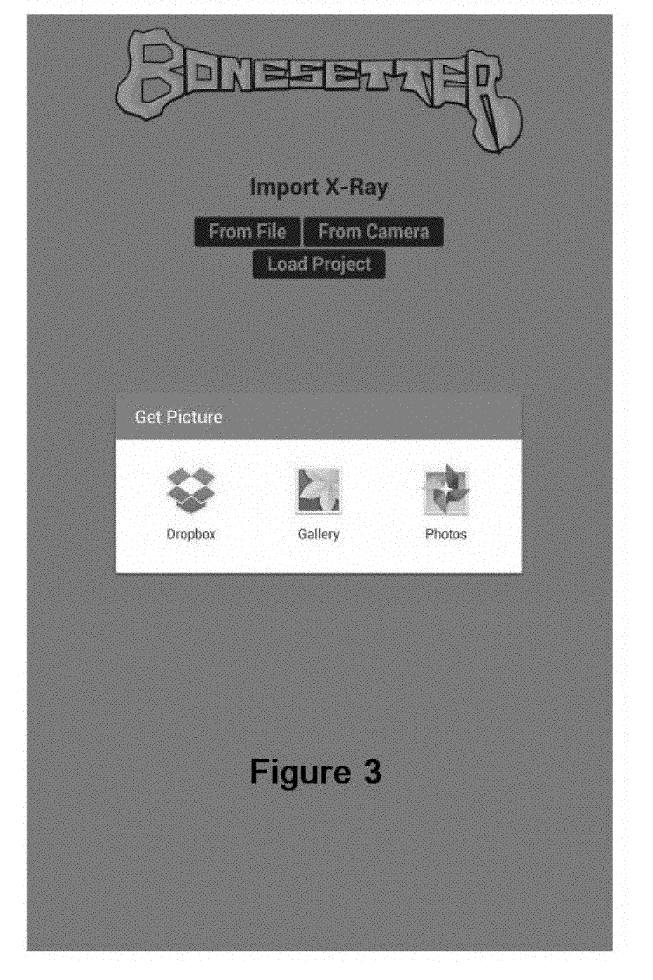

FIG. 3 is a screen shot of the GUI when user has chosen to load an x-ray from a file, in this case the mobile device allows for retrieval of a file from folders on the internal memory of the mobile device or from a storage cloud via wired or wireless connection.

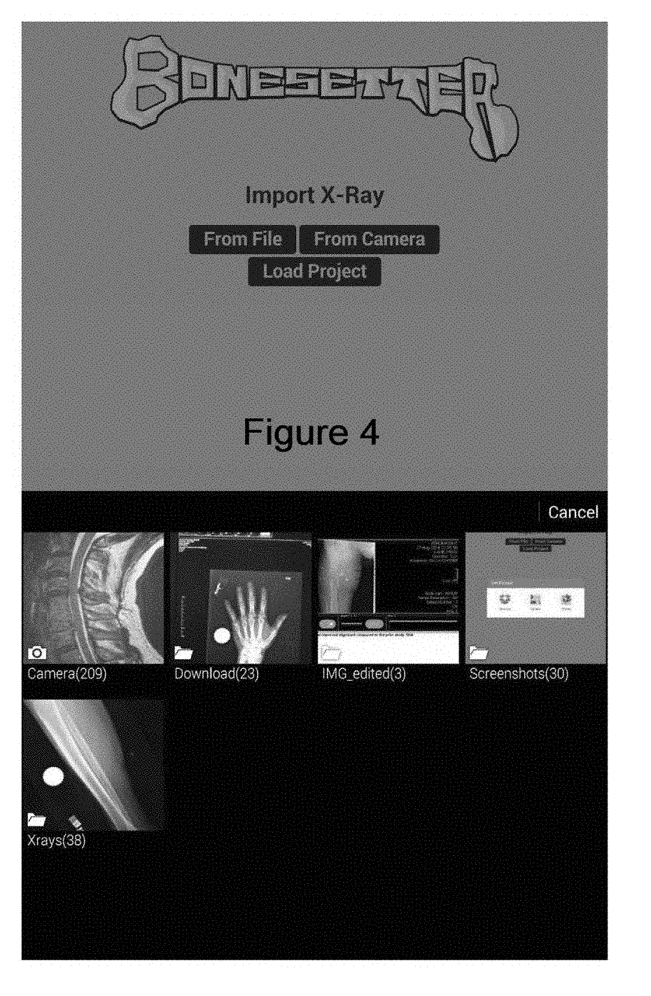

FIG. 4 is a screen shot of the GUI when user has chosen to load an x-ray from the gallery folder.

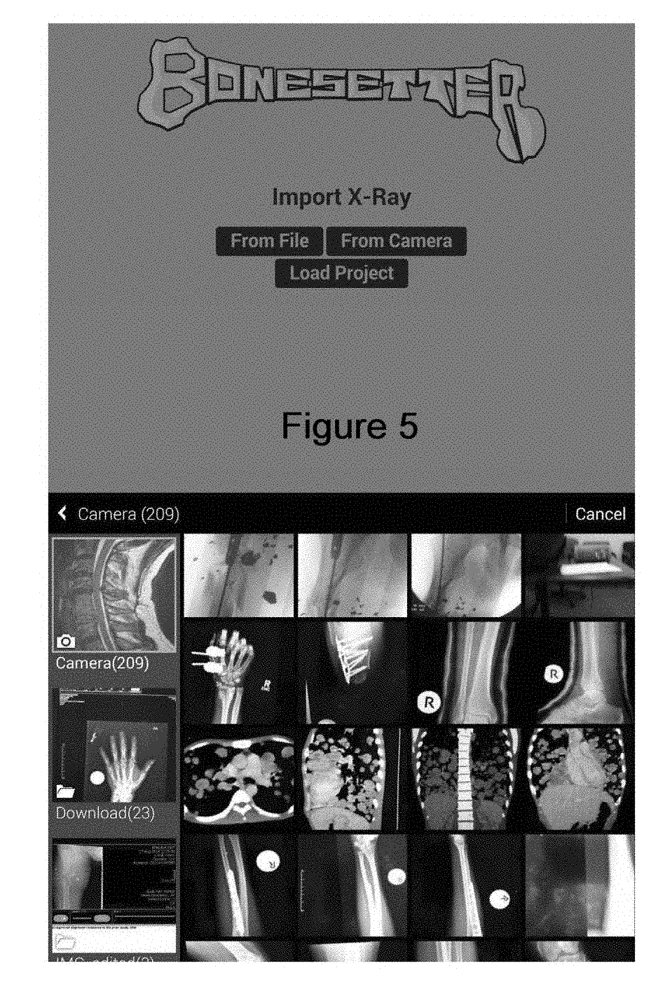

FIG. 5 is a screen shot of the GUI when user has chosen to load an x-ray from the camera subfolder.

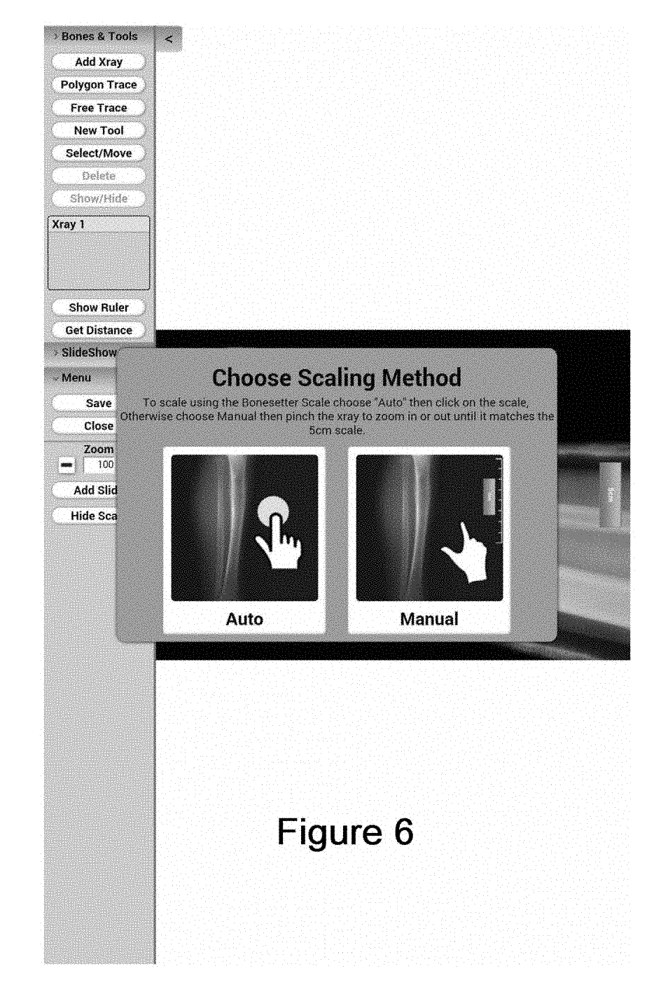

FIG. 6 is a screen shot of the GUI when user has chosen to load a specific x-ray from a specific image file. The x-ray is scaled to fit the display screen and then displayed onto the screen. Then the user is requested to choose a scaling the method, either auto scaling or manual scaling.

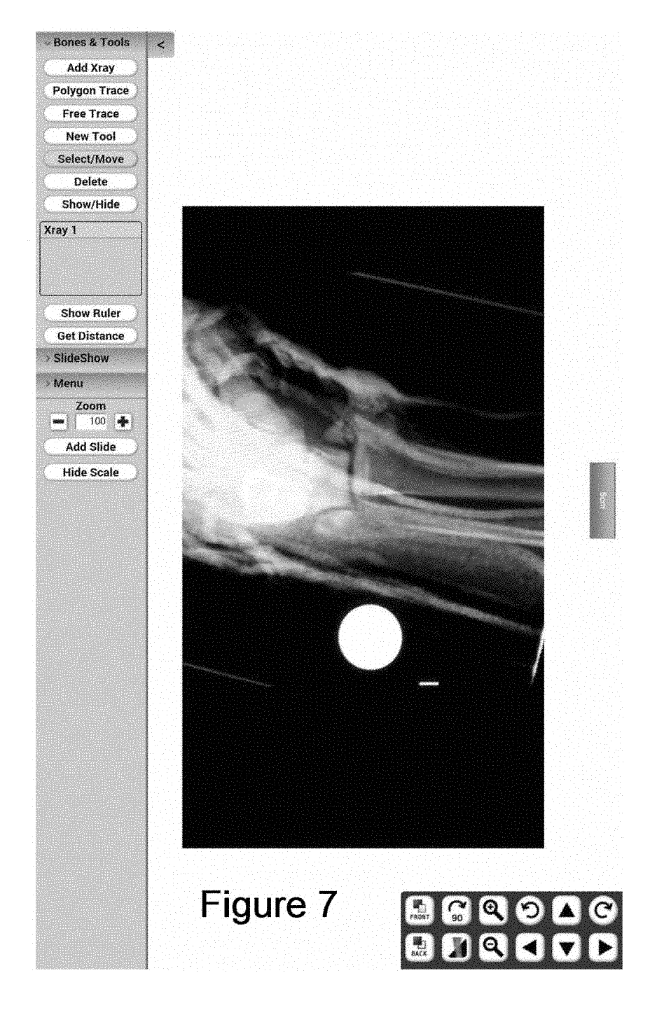

FIG. 7 is a screen shot of the GUI when user has chosen to use the auto scaling option. The user then touches in a light colored area within the scaling image (i.e. white circle) on the x-ray. The program then determines the diameter of the circle in pixels and calculates the pixel to length scaling index. As can be seen, the program has added XRAY 1 to the beginning of an image object/layer list.

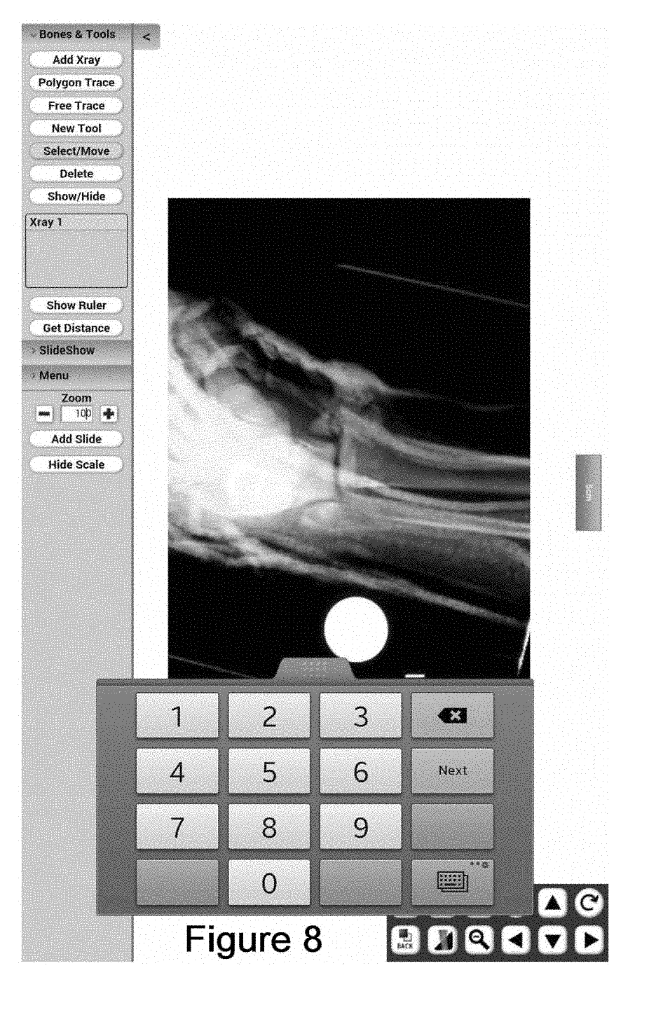

FIG. 8 is a screen shot of the GUI when user has chosen to zoom the image to better view the bones for tracing. The user is using the zoom interface on the left hand control menu or panel. selecting the zoom percentage box brings up a numeric input pad where the zoom percentage can be typed directly into the box. When the user presses next, the display of the x-ray image is immediately resized to correspond to the zoom percentage. The user may also use the zoom-in and zoom-out buttons on a quick pick button bar that can be displayed in addition to the menu on the left. More about the quick pick button bar will be discussed herein below.

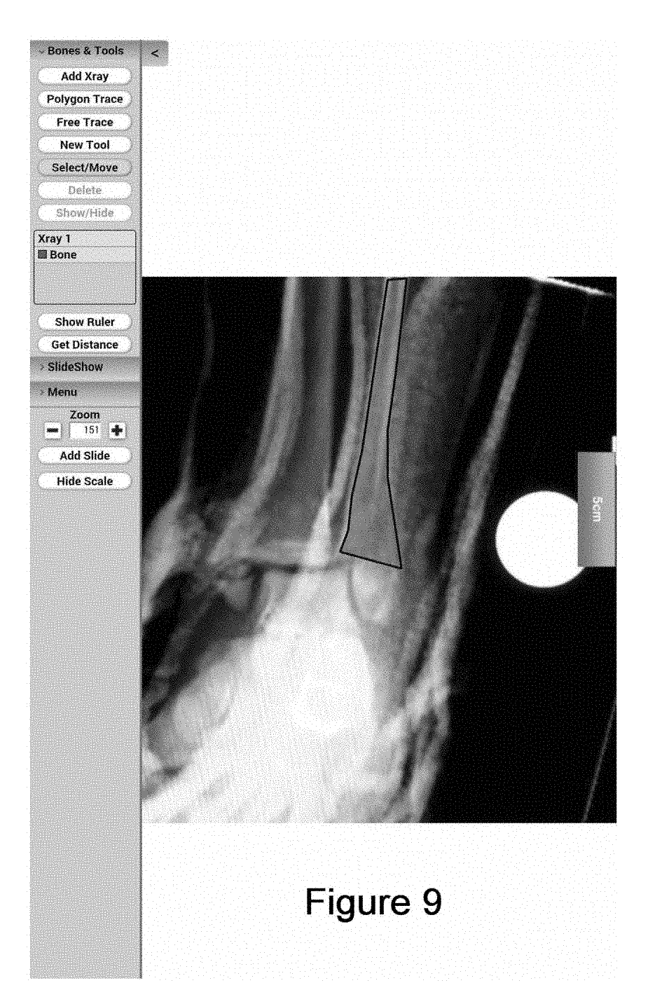

FIG. 9 is a screen shot of the GUI when user has chosen to create a polygon trace of a bone segment. The control button fora polygon trace is located in the left menu. The user clicks a point to point perimeter around the desired bone or fragment. Clicking a point close to the origin of the trace closes the perimeter and the image is filled with color. As can be seen, the program has added a red box next to the word BONE to the image object/layer list in the left control menu after XRAY1. The red box indicates the color of the bone trace overlay. The program then creates an overlay image which can be moved or rotated by dragging the image or around the screen and rotating the image directly by moving a rotation circle associated with the overlay image. The overlay image may also be moved using the up, down, right and left arrow buttons on the quick pick button bar. The overlay image may additionally be rotated using the clockwise, counterclockwise and the 90 degree rotation buttons on the quick pick button bar. See FIG. 7 for an image of the quick pick button bar. The overlay can also be created by selecting free trace button on the left menu and then directly tracing the entire perimeter of the bone or fragment.

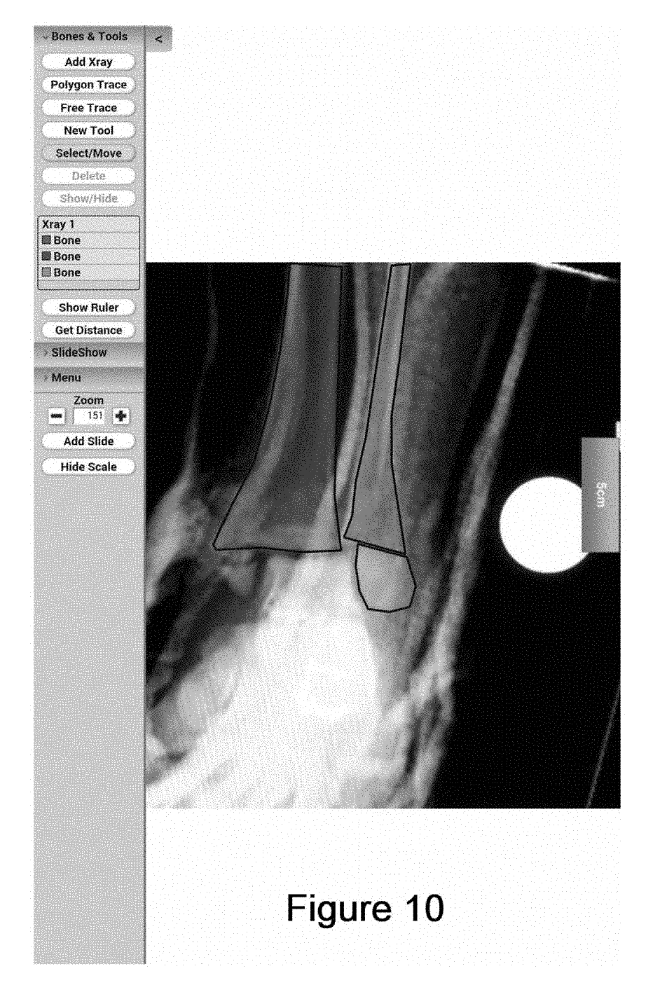

FIG. 10 is a screen shot of the GUI when user has created two more polygon traces of bone segments. As can be seen, the program has added two more BONE segment layers/objects (blue and green box) to the image object/layer list in the left control menu after the red box BONE.

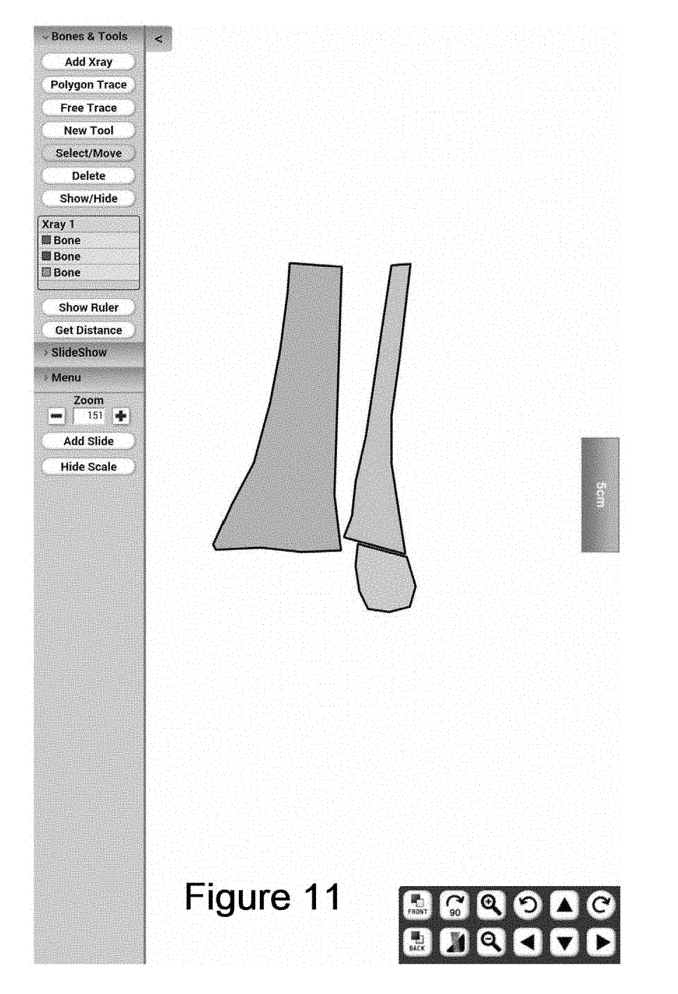

FIG. 11 is a screen shot of the GUI when user selected the XRAR 1 layer and has used the show/hide button to hide the x-ray. This leaves the overlays of the bones/fragments visible in the display image. The bone overlays may be moved and rotated to align the bones for application of implants. It should be noted that any layer can be shown/hidden or even deleted by selecting the object/layer in the list of image object/layer list and then selecting the show/hide or delete buttons.

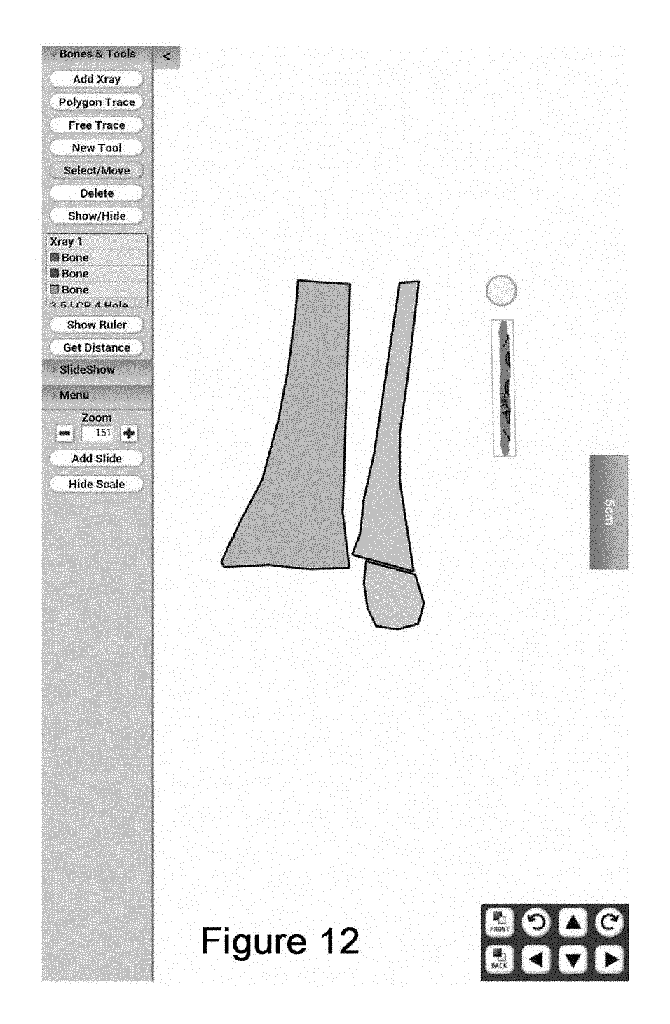

FIG. 12 is a screen shot of the GUI when user has selected the new tool button to import an implant image. A menu of implant devices is displayed and the user selects a desired and an image of the implant is scaled and inserted into the image. In this figure, the user has selected a bone plate. The image may be moved/rotated as described above.

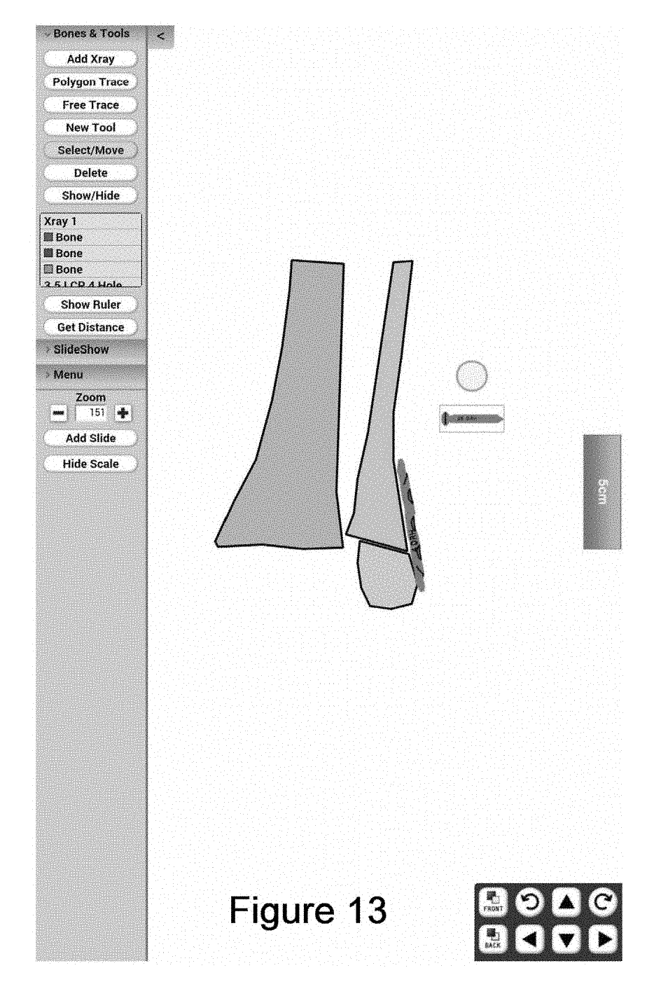

FIG. 13 is a screen shot of the GUI when user has moved the bone plate into place adjacent the bone overlays. The user then selected a 26 mm cortical screw.

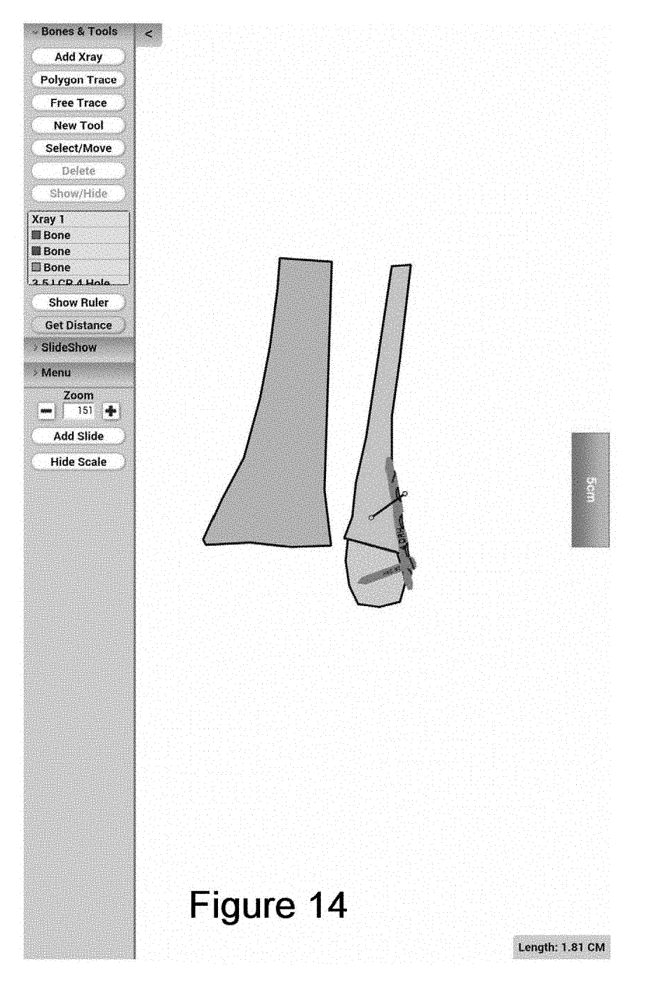

FIG. 14 is a screen shot of the GUI when user has moved the 26 mm cortical screw into place to fix the plate to one of the bone overlays. The user then used the get distance button in the left menu, chose a starting and ending point (to measure, for instance, the length of screw needed to further fix the plate to the bone. As can be seen the distance is 1.81 cm. This indicates that the next implant should be an 18 mm cortical screw.

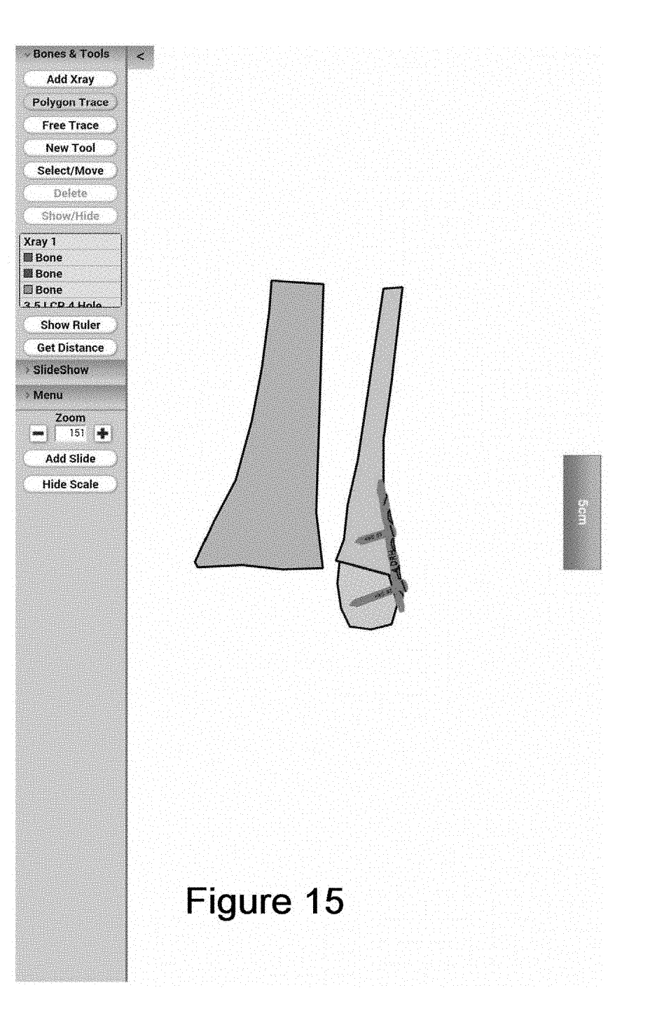

FIG. 15 is a screen shot of the GUI when user has imported and placed the 18 mm cortical screw.

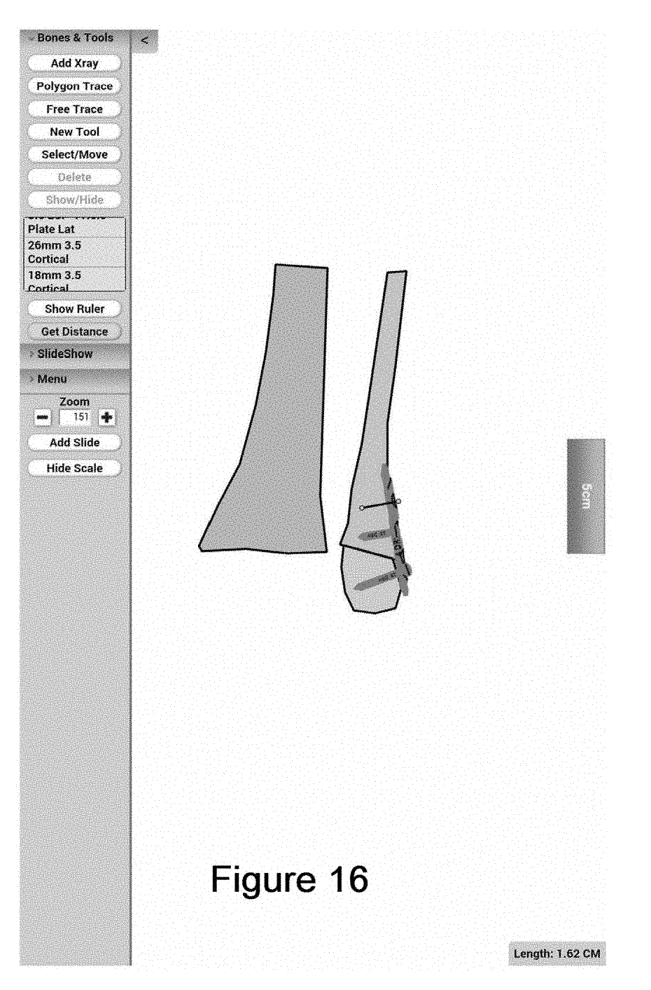

FIG. 16 is a screen shot of the GUI when user has again used the distance feature to get the length of screw needed for the top of the plate. A 16 mm cortical screw is chosen for the final fixation screw of this example.

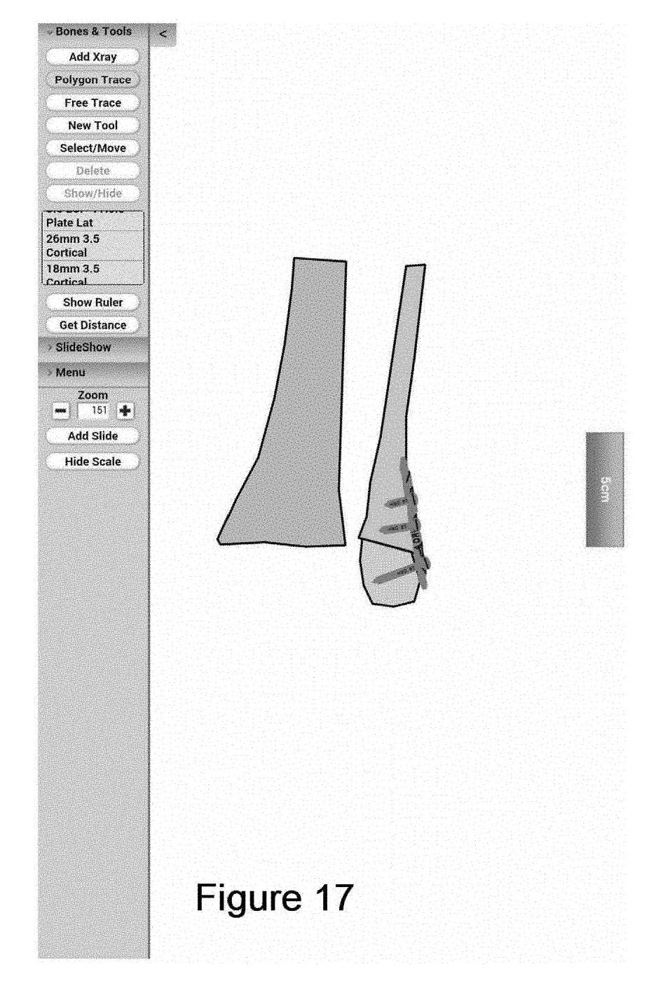

FIG. 17 is a screen shot of the GUI when user has imported and placed the final, 16 mm cortical screw. The preplanning is now completed.

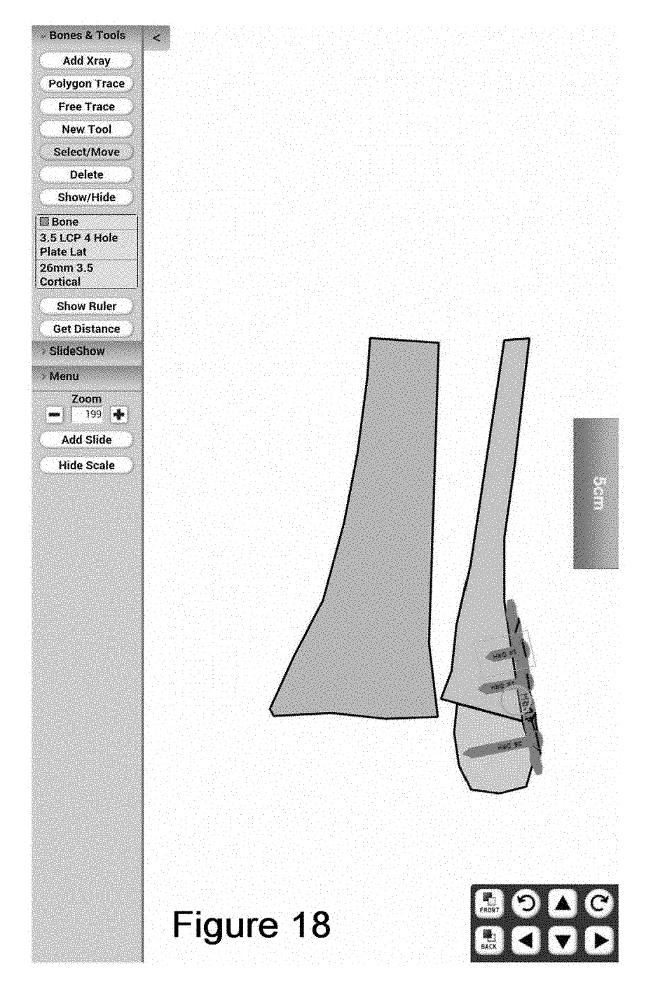

FIG. 18 is a screen shot of the GUI showing the completed virtual surgery further zoomed to 191%. The movement box and rotation circle are specifically visible in relation to one of the orthopedic implant overlay images.

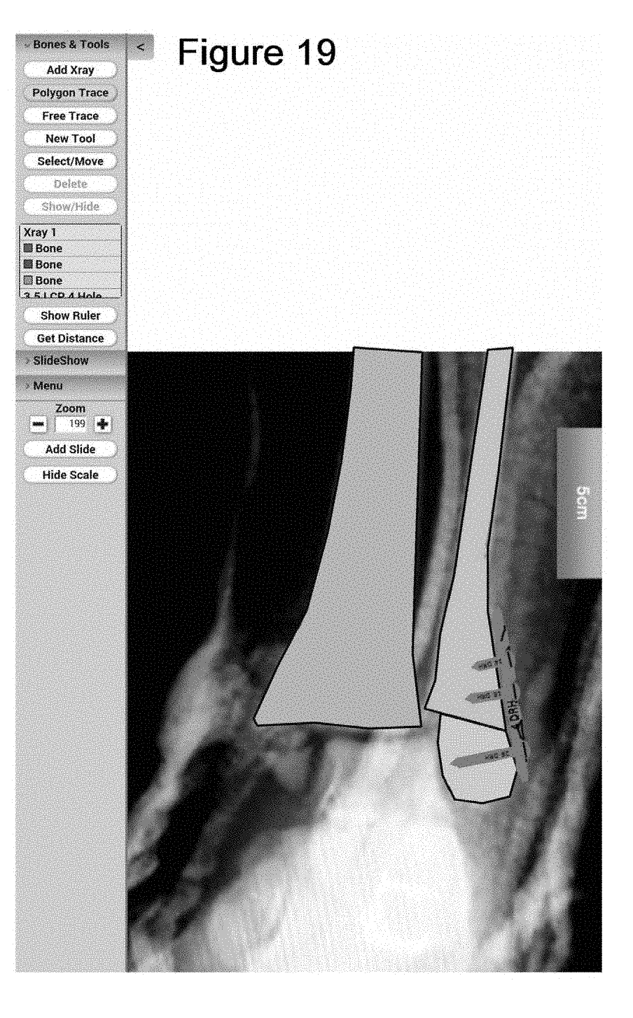

FIG. 19 is a screen shot of the GUI when the user has used the show/hide button to bring the original x-ray back into view under the bone trace and orthopedic image overlays.

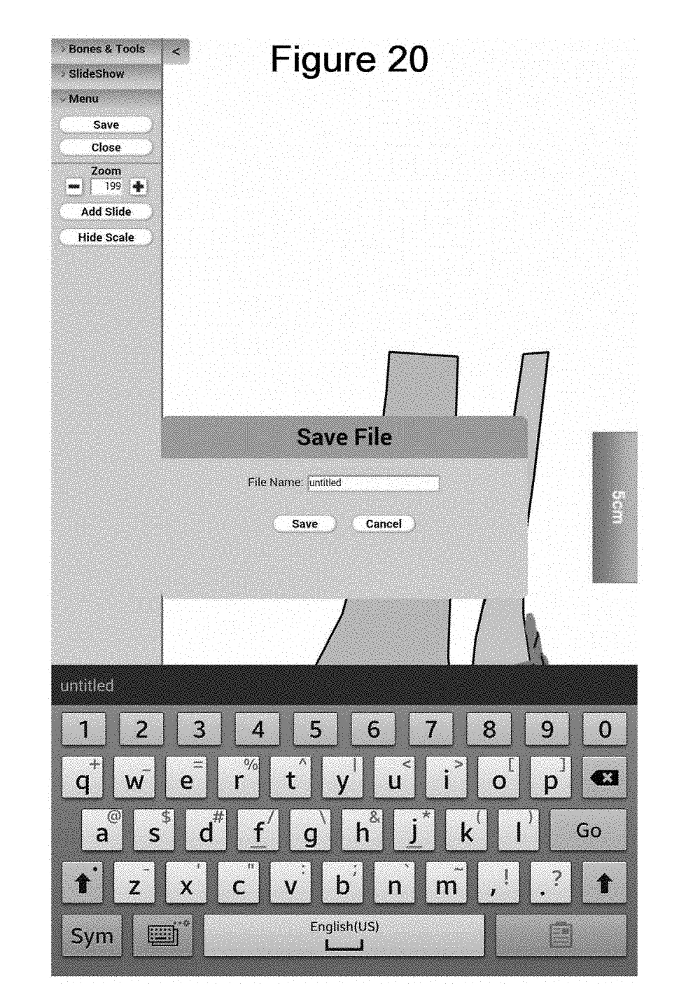

Finally, FIG. 20 is a screen shot of the GUI when the user has chosen the save button to save the project. A keypad is displayed to name the project and all of the information to recreate the project is stored to storage memory, hard disk, etc.

As can be seen, from, for instance, FIG. 20, there is a slide show button/sub-menu and an add slide button under the menu sub-menu. These are used to add a slide to the slide show, the specifics of how this is done are described herein above. The slide show button allows the user to display the slides in sequence to display the steps of the surgical plan.

There are also a show (& hide) ruler button and a hide (& show) scale bar button in the left menu. The show ruler button displays a ruler which can be used to measure distances in the image and the hide scale button allows for the blue scale bar to be hidden.

It is to be expected that considerable variations may be made in the embodiments disclosed herein without departing from the spirit and scope of this invention. Accordingly, the significant improvements offered by this invention are to be limited only by the scope of the following claims.

We claim:

1. A method of preplanning orthopedic surgery comprising:
importing an x-ray image into an electronic canvas;
scaling said x-ray image to determine the pixel to distance ratio;
creating on said canvas one or more moveable, rotatable trace overlays of bones or bone fragments visible in said x-ray;
inserting onto said canvas one or more moveable, rotatable images of orthopedic implants;
rotating and moving said trace overlays and said images of orthopedic implants into positions depicting the planned surgical outcome.

2. The method of claim 1, wherein said step of importing said x-ray image into said electronic canvas comprises importing said x-ray from a saved computer file.

3. The method of claim 1, wherein said step of importing said x-ray image into said electronic canvas comprises importing said x-ray from a camera attached to a computer.

4. The method of claim 1, wherein said step of creating said trace overlays comprises electronically tracing the perimeter of said bones or bone fragments.

5. The method of claim 4, wherein said step of tracing the perimeter of said bones or bone fragments comprises a free trace of the perimeter of said bones or bone fragments.

6. The method of claim 4, wherein said step of tracing the perimeter of said bones or bone fragments comprises a point to point polygonal trace of the perimeter of said bones or bone fragments.

7. The method of claim 1, wherein said step inserting images of orthopedic implants comprises the steps of:
selecting an orthopedic implant from a list of implants;
importing an image of said selected orthopedic implant from a database;
scaling said imported image of said selected orthopedic implant using the pixel to distance scale of the x-ray image.

8. The method of claim 1, wherein said step of scaling said x-ray image to determine the pixel to distance ratio comprises manual scaling of said image.

9. The method of claim 1, wherein said step of scaling said x-ray image to determine the pixel to distance ratio comprises automatic scaling using an image of a scale embedded into said x-ray.

10. The method of claim 9, wherein said image of a scale embedded into said x-ray is created by an x-ray opaque object of known dimensions placed the field of the x-ray as it is created.
